(19) United States
(12) Patent Application Publication
(10) Pub. No.: US 2011/0032104 A1
(43) Pub. Date: Feb. 10, 2011

(54) MEDICINE MANAGEMENT APPARATUS FOR MANAGING PATIENT'S MEDICATION PERIOD THROUGH MESSAGE TRANSMISSION

(76) Inventor: Gil-Hyeong CHO, Suwon (KR)

Correspondence Address: HARNESS, DICKEY & PIERCE, P.L.C., P.O. BOX 828, BLOOMFIELD HILLS, MI, 48303, US

(21) Appl. No.: 12/622,951

(22) Filed: Nov. 20, 2009

(30) Foreign Application Priority Data
Aug. 6, 2009 (KR) 10-2009-0072233

(51) Int. Cl. G08B 23/00; A61B 5/15; G04B 47/00

(52) U.S. Cl. 340/573.1; 600/365; 368/10; 340/309.16

(57) ABSTRACT

A medicine management apparatus comprises a medicine storage casing for storing medicines, a display unit for displaying medication times for the medicines and temperature and humidity within the medicine storage casing, an audio unit for informing the medication times on a periodic basis, a timer for setting the medication times on a periodic basis and outputting the medication times through the audio unit or the display unit, a message transmission module for transmitting a message to an administrator over a wireless communication network when there is no open signal detected when doors provided in the medicine storage casing are opened using the open signal as a medication signal, and a control unit for performing an overall control operation.

### CROSS-REFERENCE TO RELATED APPLICATIONS

This application claims the benefit and priority of Korean Patent Application No. 10-2009-0072233, filed Aug. 6, 2009. The entire disclosure of the above application is incorporated herein by reference.

### BACKGROUND OF THE INVENTION

1. Field of the Invention

The present invention relates to a medicine management apparatus, and more particularly, to a medicine management apparatus having a function of storing medicines, a function of notifying the medication times, and a function of enabling an external administrator to manage whether a patient requiring periodic medications has taken medicine over a network.

2. Background of the Related Art

In general, numerous medicines and instruments are being used according to the symptoms and uses of patients. Storage conditions differ depending on the types or uses of the medicines and the instruments. It is necessary to manage medication for a patient who must periodically take medicine.

The storage conditions can include, for example, drink types for cold storage and hot storage. For example, herb medicines are extracted in a liquid phase, filled in the package, stored in a cold state (for example, 4 to 8° C.), and then heated and used. XALATAN EYE DROPS (for example, 2 to 8° C. storage), ANDRIOL SOFT CAPSULES (2 to 3° C. storage), GYNOFLOR VAG. TABS (4 to CC storage), and so on require cold storage. Eye drops, such as DECROL EYE DROPS and OCUCHLORAM EYE DROPS, require storage lower than 10° C. or less. CEROXEN TABS (i.e., arthritis remedies), LISOLBIN SOFT CAPSULES (i.e., hyperlipidemia remedies), DONGWHA THYMIDIN CAPSULES (i.e., viral disease remedies), etc. require dry, cold, and darkroom storage.

As described above, medicines are classified into medicines for hot storage, medicines for cold storage, and medicines for room-temperature storage according to the types. However, conventional medicine management apparatuses are used only for a hot storage, a cold storage, and a room-temperature storage, respectively.

To solve the problem, Korean Utility Model Registration No. 20-0335370 discloses a medicine management apparatus having a room-temperature storage room, a cold storage room, a hot storage room, and an ultraviolet disinfecting room integrated therein.

However, the medicine management apparatus disclosed in Korean Utility Model Registration No. 20-0335370 is advantageous in that it can be used in various ways according to the types and storage conditions of medicines, but is disadvantages in that it does not have additional effects other than the medicines storage function. That is, the medicine management apparatus has only limited functions (for example, a patient's medication times).

Furthermore, there are recently being disclosed technologies equipped with remote medical systems for patients and configured to inform the patients of the medication times with the help of a developed network.

However, the conventional technologies are configured to inform the patients of only medication times and medication methods, but are not configured to manage whether patients (for example, hypertensives, diabetic patients, paralytics, and imbecility patients) periodically take medicine. Furthermore, a medicine management apparatus capable of managing whether patients have taken medicine using an apparatus for storing medicines has not yet been developed.

### SUMMARY OF THE INVENTION

Accordingly, some embodiments of the present invention have been made in view of the above problems, and it is an object of the present invention to provide a medicine management apparatus capable of storing medicines according to the storage conditions of the medicines.

Another object of the present invention is to provide a medicine management apparatus equipped with a blood sugar measuring instrument, enabling a diabetic patient himself to check his blood sugar.

Yet another object of the present invention is to provide a medicine management apparatus which is capable of assisting a patient who must periodically take medicine by informing the patient of the medication time in the form of an audio or video signal.

Further yet another object of the present invention is to provide a medicine management apparatus which is capable of managing a patient at a remote place by determining whether the patient has taken medicine based on signals detected when the doors of the medicine management apparatus are opened and informing an administrator at the remote place of the determination result.

To achieve the above objects, according to the present invention, there is provided a medicine management apparatus for managing a patient's medication period through message transmission, the apparatus comprising a medicine storage casing for storing medicines, a display unit for displaying medication times for the medicines and temperature and humidity within the medicine storage casing, an audio unit for informing the medication times on a periodic basis, a timer for setting the medication times on a periodic basis and outputting the medication times through the audio unit or the display unit, a Short Message Service (SMS) transmission module for transmitting a SMS message to an administrator over a wireless communication network when there is no open signal detected when doors provided in the medicine storage casing are opened using the open signal as a medication signal, and a control unit for performing an overall control operation.

The medicine management apparatus may further comprise a wired communication module for transmitting an emergency signal to a management server over a wired communication network when there is no open signal using the open signal as the medication signal.

When there is no open signal of the doors, the audio unit or the display unit preferably is controlled to output a medication notification signal again based on the period set in the timer in response to a periodic signal.

The SMS transmission module and the wired communication module preferably may transmit the SMS message and an emergency signal to the administrator and a management server, respectively, when there is no open signal although the medication notification signal is outputted again by a predetermined number of times in response to the periodic signal.

The medicine storage casing preferably is partitioned into a hot storage room, a cold storage room, and a room-temperature storage room with a barrier rib interposed therebetween. Temperature and humidity within each of the storage rooms preferably are controlled by the control unit.

The timer preferably comprises a first timer, a second timer, and a third timer configured to set the respective medication times of the medicines stored in the hot storage room, the cold storage room, and the room-temperature storage room.

The medicine management apparatus may further comprise a blood sugar measuring instrument for enabling a diabetic patient himself to measure his blood sugar.

A blood sugar value measured by the blood sugar measuring instrument may be transmitted to the administrator and the management server through the message transmission module and the wired communication module, respectively.

The medicine management apparatus may further comprise an urgent button unit disposed on one side of the medicine management apparatus and configured to transmit an urgent signal according to the patient's urgent situation to a management server or an administrator over wired/wireless communication networks.

### BRIEF DESCRIPTION OF THE DRAWINGS

Further objects and advantages of the invention can be more fully understood from the following detailed description taken in conjunction with the accompanying drawings in which:

FIG. 1 is a block diagram showing the internal construction of a medicine management apparatus according to an embodiment of the present invention;

FIG. 2 is a schematic diagram showing a system for transmitting an emergency signal to a management server and an administrator through SMS or over a wired communication network using the medicine management apparatus according to an embodiment of the present invention;

FIG. 3 is a schematic diagram showing a system for, when an urgent situation is generated, transmitting an urgent signal to the management server and the administrator through SMS or over a wired communication network using the medicine management apparatus according to an embodiment of the present invention; and

FIG. 4 is a schematic diagram showing a system for transmitting a measured blood sugar value to the management server and the administrator through SMS or over a wired communication network using the medicine management apparatus according to an embodiment of the present invention.

### DETAILED DESCRIPTION OF THE PREFERRED EMBODIMENT

Hereinafter, the constructions and acting effects of a medicine management apparatus capable of managing a patient's medication period through message transmission (hereinafter referred to as a ‘medicine management apparatus’) according to some embodiments of the present invention are described in detail with reference to the accompanying drawings.

The terminology ‘message’ used in the present invention is a concept including, in general, Short Message Service (SMS, short message), Long Message Service (LMS, long message), and Multi-media Message Service (MMS, text and images). It is to be noted that in the following embodiments, SMS transmission is described as an example, and a ‘message transmission module’ described in the claims refers to an SMS transmission module in the SMS, an LMS transmission module in the LMS, and an MMS transmission module in the MMS.

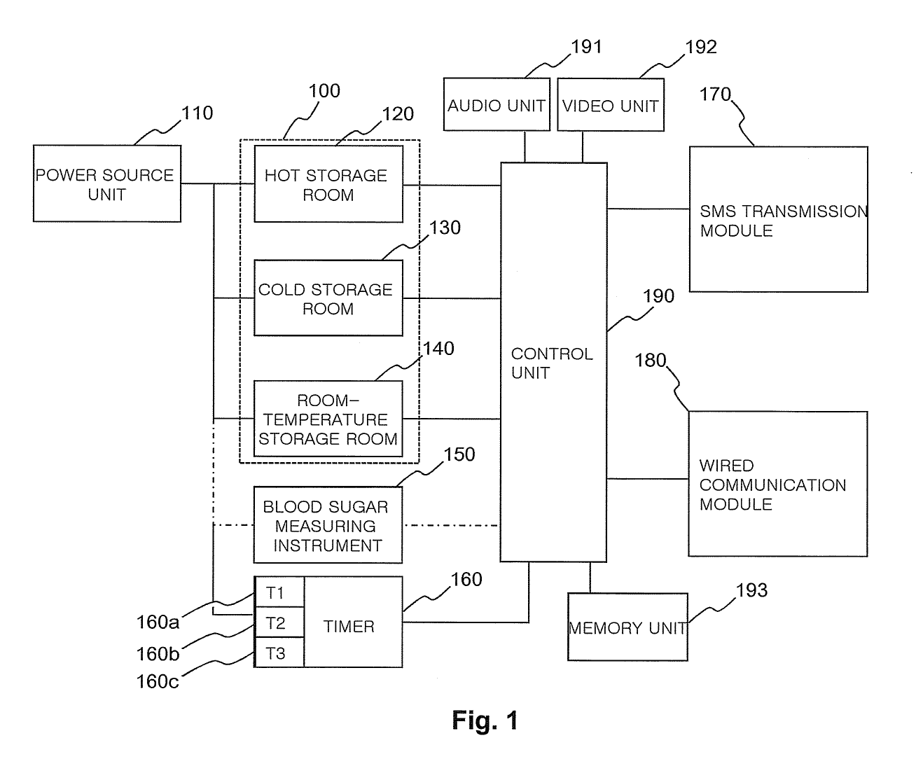

FIG. 1 is a block diagram showing the internal construction of a medicine management apparatus according to an embodiment of the present invention.

As shown in FIG. 1, the medicine management apparatus according to the present embodiment includes a power source unit 110, a medicine storage casing 100 consisting of a hot storage room 120, a cold storage room 130, and a room-temperature storage room 140, a blood sugar measuring instrument 150, a timer 160, an SMS transmission module 170, a wired communication module 180, a control unit 190, an audio unit 191, a video unit 192, and a memory unit 193.

The medicine storage casing 100 is partitioned into the hot storage room 120, the cold storage room 130, and the room-temperature storage room 140. The storage rooms 120, 130, and 140 can be isolated from each other by a barrier rib that is made of adiabetic materials and configured to prevent the transfer of heat therebetween. Furthermore, the storage rooms can be equipped with respective independent doors and can be placed in respective partitioned shelves up and down or left and right in the form of a drawer type.

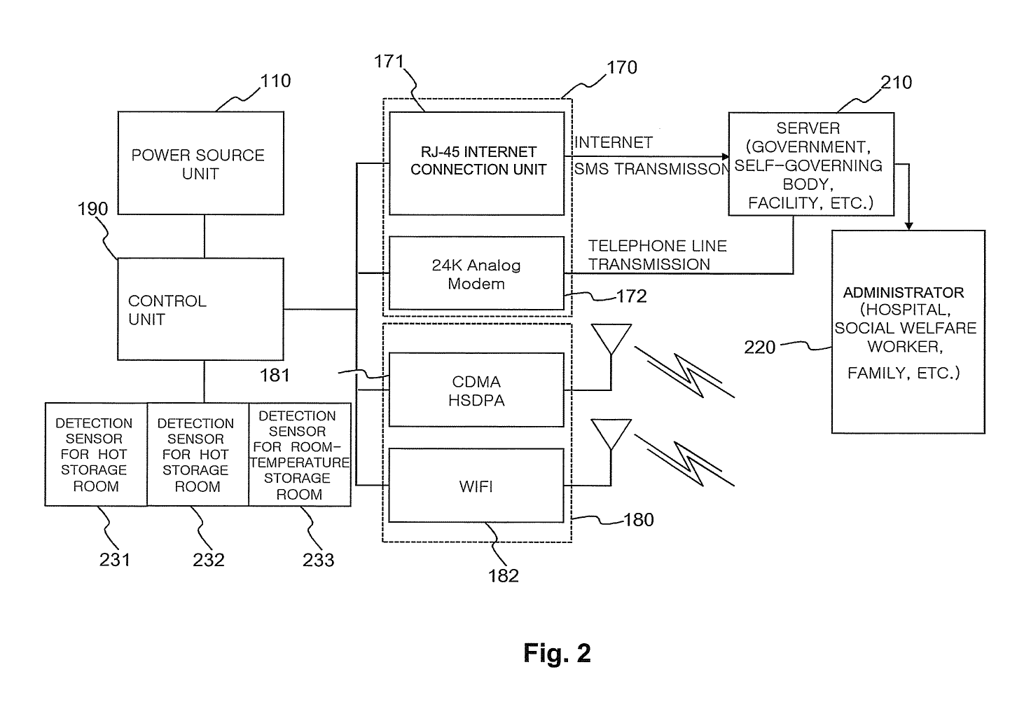

Temperature and humidity of each of the hot storage room 120, the cold storage room 130, and the room-temperature storage room 140 can be independently set by the control unit 190. Independent power sources can be supplied to the storage rooms. Open sense sensors (refer to 231 to 233 of FIG. 2) for sensing signals generated when the doors of the storage rooms 120, 130, and 140 are opened are respectively mounted on the doors of the storage rooms 120, 130, and 140.

The blood sugar measuring instrument 150 is configured to enable a patient himself to measure his blood sugar. The blood sugar measuring instrument 150 may preferably include a string connection wire unit configured to have a test strip connected thereto, a blood sugar measuring unit configured to include an active electrode and an inactive electrode, an analog-digital converter configured to convert an analog value measured by the blood sugar measuring unit into a digital signal, a microprocessor configured to measure blood sugar, and a communication unit configured to transmit the measured blood sugar value over wired/wireless communication networks. The blood sugar measuring instrument for transmitting a measured blood sugar value over the wired/wireless communication networks is known in the art, and a detailed description thereof is omitted.

The timer 160 may include a first timer 160a for the hot storage room 120, a second timer 160b for the cold storage room 130, and a third timer 160c for the room-temperature storage room 140.

The first to third timers 160a to 160c are configured to set the medication times of medicines stored in the respective storage rooms 120 to 140. Furthermore, if a certain period of time elapses from a medication time, a periodic value for outputting a medication notification signal again in order to repeatedly inform the medication time is set in each of the first to third timers 160a to 160c.

The memory unit 193 stores operating programs of the medicine management apparatus. The memory unit 193 further stores messages which will be transmitted to a management server or an administrator at a remote place via the SMS transmission module 170 and the wired communication module 180 in the form of a table.

The SMS transmission module 170 and the wired communication module 180 are of wired/wireless communication units for transmitting the messages, stored in the memory unit 193, to the management server or the administrator at the remote place while operating in conjunction with the timer 160 under the control of the control unit 190.

The control unit 190 is a main controlled that is responsible for an overall control operation of the medicine management apparatus. The power source unit 110 supplies a power source. The audio unit 191 can be, for example, a typical speaker for informing the medication times through voice or for giving an alarm. The video unit 192 can be, for example, a Liquid Crystal Display (LCD) for displaying the medication times, temperature and humidity of each storage room, the current time, etc.

Configurations for transmitting a medication notification signal, an emergency signal, and an urgent signal using the medicine management apparatus constructed as above according to the present invention are described below with reference to FIGS. 1 to 4.

First, signals described in the present invention are defined as follows.

A door open signal refers to a signal detected by each of the open detection sensors 231 to 233 when the respective doors of the storage rooms 120 to 140 are opened. The door open signal is used to determine whether a patient has taken medicine on a predetermined time at a remote place.

A medication notification signal is used for the medicine management apparatus to directly inform a patient that a medication time has reached.

An emergency signal is transmitted to the management server and the administrator at a remote place when a patient has not taken medicine on a predetermined time. The emergency signal is transmitted when the door has not been opened although the medication notification signal has been repeatedly transmitted several times according to a period set in the timer on the basis of a door open signal. The management server or the administrator that has received the emergency signal determines that a patient has not taken medicine on a predetermined time and takes appropriate measures.

An urgent signal is used to inform the management server or the administrator at a remote place that a patient is urgent by pressing an urgent button unit 240 provided at a specific location of the medicine management apparatus.

Referring to FIG. 1 medication time values of medicines stored in the storage room 120 to 140 are set in the first to third timers 160a to 160c, respectively. A patient or the family of the patient who is near the medicine management apparatus is informed of the set medication times through the audio unit 191 and the video unit 192 based on messages stored in the memory unit 193 under the control of the control unit 190. The patient can take medicine on the predetermined times based on the medication notification signals.

FIG. 2 is a schematic diagram showing a system for transmitting an emergency signal to a management server and an administrator through SMS or over a wired communication network using the medicine management apparatus according to an embodiment of the present invention.

If, although a medication notification signal has been sent, a corresponding medicine storage room has not been opened, a corresponding one of the open detection sensors 231 to 233 repeatedly sends the medication notification signal according to a predetermined period.

Here, if the corresponding one of the open detection sensors 231 to 233 detects that the corresponding medicine storage room has not yet been opened in response to the repeatedly transmitted medication notification signal, the control unit 190 transmits an emergency signal to the management server 210 through a wired communication module 170 (i.e., an RJ-45 Internet connection unit 171 or a 24K analog modem 172). The management server 210 transfers the received emergency signal to the administrator 220 such that the administrator 220 can check the urgent situation of the patient. Alternatively, the control unit 190 may directly send SMS, corresponding to the emergency signal, to the administrator 220 through an SMS transmission module 180 (i.e., CDMA and HSDPA 181 or a WiFi module 182) such that the administrator 220 can check the urgent situation of the patient.

The management server 210 may correspond to a medical service-related department of a central government, a medical service-related department of a self-governing body, a private medical service-related company, etc. The administrator 220 may correspond to a person who is involved in a hospital that is in full charge of a patient, a social welfare worker, or a patient's relatives.

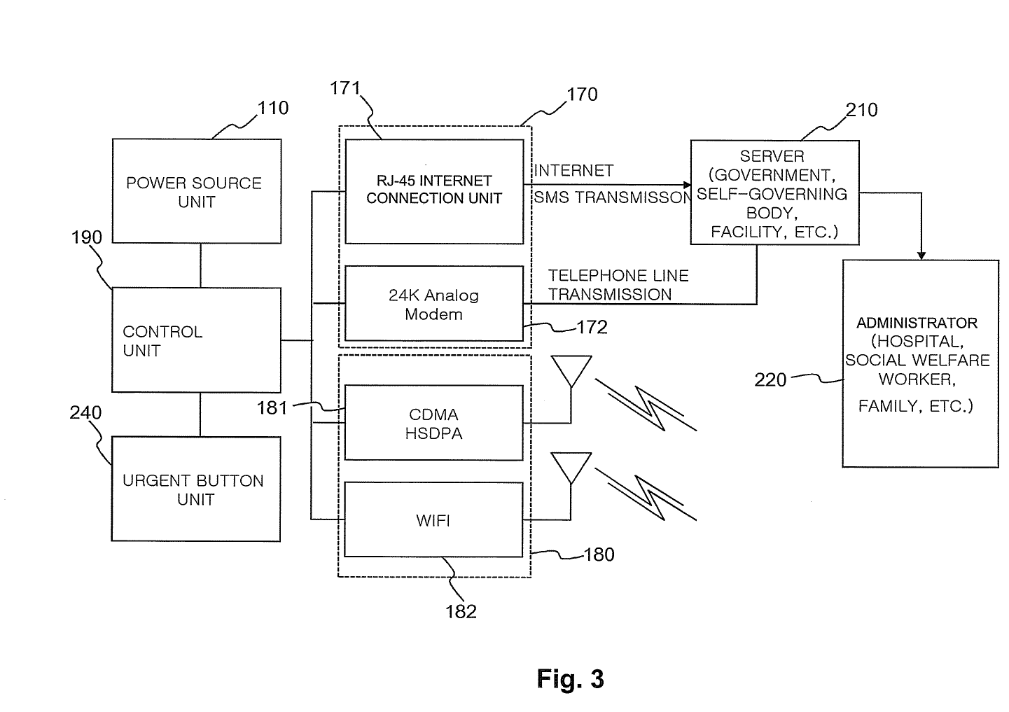

FIG. 3 is a schematic diagram showing a system for, when an urgent situation is generated, transmitting an urgent signal to the management server and the administrator through SMS or over a wired communication network using the medicine management apparatus according to an embodiment of the present invention.

In the case where a patient is in an urgent situation, when a patient or the family of the patient presses the urgent button unit 240 provided at a specific location of the medicine management apparatus, the control unit 190 transmits an emergency signal to the management server 210 through the wired communication module 170 (i.e., the RJ-45 Internet connection unit 171 or the 24K analog modem 172). The management server 210 transfers the received emergency signal to the administrator 220 such that the administrator 220 can check the urgent situation of the patient and take appropriate measures. Alternatively, the control unit 190 may directly transmit SMS, corresponding to the emergency signal, to the administrator 220 through the SMS transmission module 180 (i.e., the CDMA and HSDPA 181 or the WiFi module 182) such that the administrator 220 can check the urgent situation of the patient and take appropriate measures.

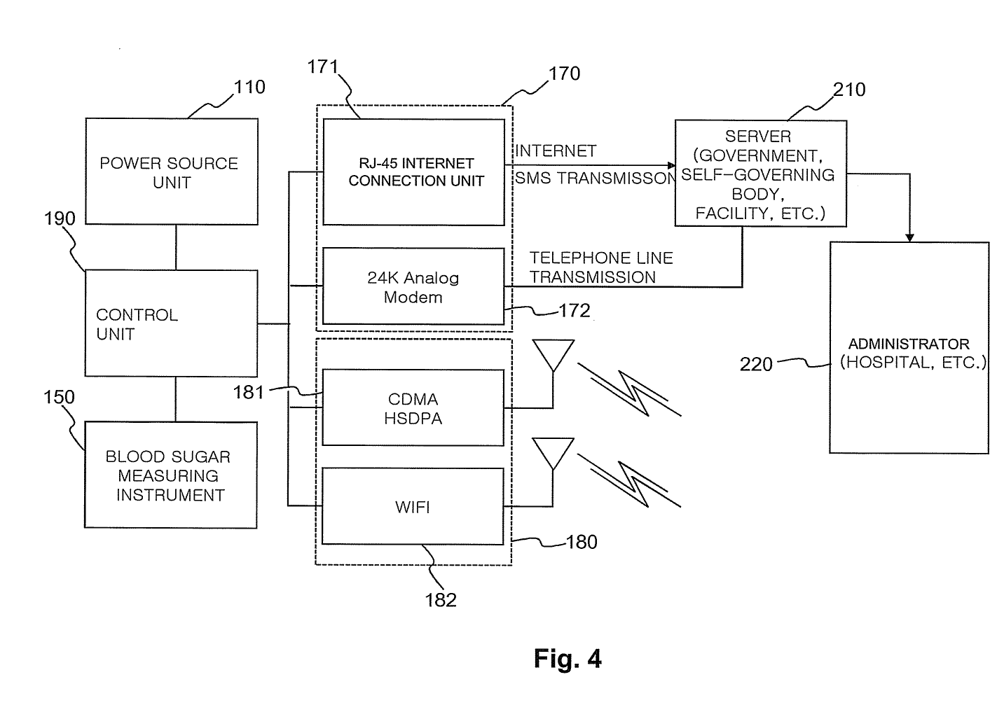

FIG. 4 is a schematic diagram showing a system for transmitting a measured blood sugar value to the management server and the administrator through SMS or over a wired communication network using the medicine management apparatus according to an embodiment of the present invention.

The blood sugar measuring instrument 150 is a blood sugar measuring instrument enabling a patient himself to measure his blood sugar. The blood sugar measuring instrument 150 includes a string connection wire unit configured to have a test strip connected thereto, a blood sugar measuring unit configured to include an active electrode and an inactive electrode, an analog-digital converter configured to convert an analog value measured by the blood sugar measuring unit into a digital signal, a microprocessor configured to measure blood sugar, and a communication unit configured to transmit the measured blood sugar value over wired/wireless communication networks.

When a patient measures his blood sugar using the blood sugar measuring instrument 150, the measured blood sugar value, together with the patient's personal history, is transmitted to the management server 210 through the wired communication module 170 (i.e., the RJ-45 Internet connection unit 171 or the analog modem 172) under the control of the control unit 190. The management server 210 transfers the measured blood sugar value and the patient's personal history to the administrator 220 such that the administrator 220 can check the patient's urgent situation and take appropriate measures. Alternatively, the control unit 190 may directly transmit the measured blood sugar value, together with the patient's personal history, to the administrator 220 through the SMS transmission module 180 (i.e., the CDMA and HSDPA 181 or the WiFi module 182) in the form of SMS such that the administrator 220 can check the patient's urgent situation and take appropriate measures.

Here, the management server 210 may correspond to a medical service-related department of a self-governing body, and the administrator 220 may correspond to a person who is involved in a hospital that is in full charge of the patient.

While some exemplary embodiments of the present invention have been described, the present invention is not to be restricted by the embodiments, but only by the appended claims. It is to be appreciated that those skilled in the art can change or modify the embodiments in various ways without departing from the scope and spirit of the present invention.

As described above, according to the present invention, medicines can be stored according to storage conditions based on the types of the medicines. The medicine management apparatus is equipped with the blood sugar measuring instrument that enables a patient himself to measure his blood sugar. Accordingly, a diabetic patient himself can check his blood sugar. Medication times can be provided to a patient who must periodically take medicine through an audio or video signal. Furthermore, whether a patient has taken medicine is determined based on signals detected when the doors of the medicine management apparatus are opened, and an administrator at a remote place is informed of the determination results. Accordingly, the patient can be managed at the remote place.

What is claimed is:

1. A medicine management apparatus for managing a patient's medication period through message transmission, the apparatus comprising:
a medicine storage casing for storing medicines;
a display unit for displaying medication times for the medicines and temperature and humidity within the medicine storage casing;
an audio unit for informing the medication times on a periodic basis;
a timer for setting the medication times on a periodic basis and outputting the medication times through the audio unit or the display unit;
a message transmission module for transmitting a message to an administrator over a wireless communication network when there is no open signal detected when doors provided in the medicine storage casing are opened using the open signal as a medication signal; and
a control unit for performing an overall control operation of the medicine management apparatus.

2. The medicine management apparatus as claimed in claim 1, further comprising a wired communication module for transmitting an emergency signal to a management server over a wired communication network when there is no open signal using the open signal as the medication signal.

3. The medicine management apparatus as claimed in claim 1, wherein the control unit controls the audio unit or the display unit such that the audio unit or the display unit outputs a medication notification signal again based on the period set in the timer in response to a periodic signal when there is no open signal.

4. The medicine management apparatus as claimed in claim 3, wherein the message transmission module and the wired communication module transmit the message and an emergency signal to the administrator and a management server, respectively, when there is no open signal although the medication notification signal is outputted again by a predetermined number of times in response to the periodic signal.

5. The medicine management apparatus as claimed in claim 1, wherein:
the medicine storage casing is partitioned into a hot storage room, a cold storage room, and a room-temperature storage room with a barrier rib interposed therebetween, and temperature and humidity within each of the storage rooms are controlled by the control unit.

6. The medicine management apparatus as claimed in claim 5, wherein the timer comprises a first timer, a second timer, and a third timer configured to set the respective medication times of the medicines stored in the hot storage room, the cold storage room, and the room-temperature storage room.

7. The medicine management apparatus as claimed in claim 1, further comprising a blood sugar measuring instrument for enabling a diabetic patient himself to measure his blood sugar.

8. The medicine management apparatus as claimed in claim 7, wherein a blood sugar value measured by the blood sugar measuring instrument is transmitted to the administrator and the management server through the message transmission module and the wired communication module, respectively.

9. The medicine management apparatus as claimed in claim 1, further comprising an urgent button unit disposed on one side of the medicine management apparatus and configured to transmit an urgent signal according to the patient's urgent situation to a management server or an administrator over wired/wireless communication networks.
